(19) United States
(12) Patent Application Publication
(10) Pub. No.: US 2021/0393163 A1
(43) Pub. Date: Dec. 23, 2021

(54) DEVICE AND METHODS FOR COLLECTING AND PROCESSING ANALYTES IN AIR/BREATH

(71) Applicant: Graham Biosciences LLC, McLean, VA (US)

(72) Inventor: David Graham, Beltsville, MD (US)

(21) Appl. No.: 17/343,316

(22) Filed: Jun. 9, 2021

Related U.S. Application Data
(60) Provisional application No. 63/036,928, filed on Jun. 9, 2020.

(51) Int. Cl. A61B 5/097; G01N 1/22; G01N 33/497

(52) U.S. Cl. CPC A61B 5/097; G01N 33/497; G01N 1/22

(57) ABSTRACT

The present invention relates to a device for the collection of breath and/or air and the processing and analysis of analytes contained therein. More particularly, the present invention relates to a method of collecting exhaled breath and/or air and processing and analyzing one or more analytes contained therein.

This application claims priority to U.S. Provisional Patent Application No. 63/036,928 filed on Jun. 9, 2020, which is hereby incorporated by reference in its entirety.

### FIELD OF INVENTION

The present invention relates to a device for the collection of one or more gases, including exhaled breath and/or air and the processing and analysis of analytes contained therein. More particularly, the present invention relates to a method of collecting exhaled breath and/or air and processing, detecting and analyzing one or more analytes contained therein.

### BACKGROUND

The detection, identification and quantification of various analytes in biological fluids is an important aspect of diagnosis, treatment and surveillance in a public health context. Important analytes are also be present in exhaled breath. The presence of such analytes, however, depends on practical methods for sampling breath and analytes contained therein. The sampling of breath in a practical manner, however, is limited with current technologies.

Collection and condensation of gases/vapors, including exhaled breath, at the time of collection requires significant energy input and results in the loss of volatile analytes. As the largest component of exhaled breath, for example, is water vapor, most energy is spent on cooling and condensing water vapor, which has a very high thermal capacity. Most volatile analytes in exhaled breath are adsorbed in equilibrium with water vapor, so a significant proportion of volatiles are lost by these types of collection methods. Because these methods are open systems, additional loss of volatile analytes is expected. Many electronic detectors also require breath to be adjusted in humidity levels so that humidity does not interfere with the detection of compounds in air/breath.

In other methods, breath is collected into a bag. Sample collection into bags, without condensing the collected gas, yields large, impractical volumes that are difficult to handle and transport and exposes analytes to conditions that lead to analyte degradation and loss (adsorption to inner surfaces of the bag).

Each of these collection methods are static and require external power for any type of analysis to be performed.

The present invention also provides a device that collects and preserves analytes contained in breath or air in a convenient, practical, low-volume manner that minimizes the loss of volatile analytes without external energy input to the device. The present invention also provides methods for the processing of these analytes, including volatile analytes, such as volatile organic compounds, that are susceptible to loss in existing prior art methods of collection and processing of exhaled breath.

### BRIEF DESCRIPTION OF THE DRAWINGS

The accompanying drawings exemplify the present invention and are not intended to limit the present invention as disclosed herein. Some or all of the figures may be schematic representations for purposes of illustration and do not necessarily depict the actual relative sizes or locations of the elements shown.

FIG. 1 schematically depicts one embodiment of a breath collection/volatile-analyte preservation device.

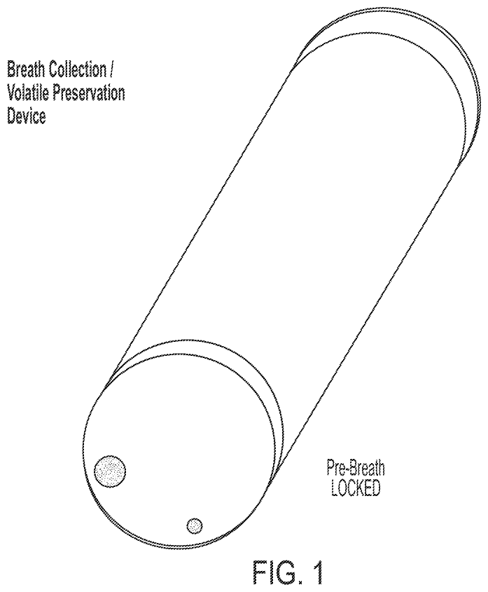

FIG. 2 schematically depicts a front view of one embodiment of a breath collection/volatile-analyte preservation device in an eight (8) chamber configuration and three valve positions. In Position 1, two valve from the collection chamber to the exterior are shown in the closed position. The intake is depicted as the larger of the two valves and the outlet valve is depicted as the smaller valve. A valve from the collection chamber to the central core (both valve and central core are not shown in this figure) is also in the closed position. Position 2 shows both valves open to the exterior of the device. In Position 2, the valve from the collection chamber to the central core (not shown) remains in the closed position. Position 3 shows both valves to the exterior of the device in the closed position. The valve from the collection chamber to the central core (not shown) is now in the open position. The central-core valve is depicted schematically in FIG. 7.

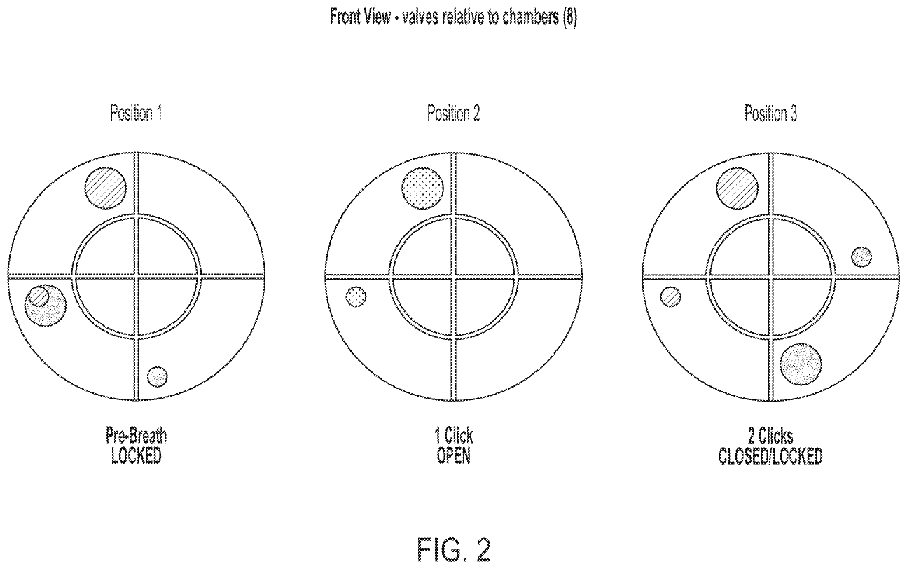

FIG. 3 schematically depicts a cut away revealing core relative to chamber of one embodiment of a breath collection/volatile-analyte preservation device. The front of the device is depicted graphically as an eight (8) chamber device. The eight chambers are not depicted in the body of the device to more clearly show the central core. The front of the device also depicts a humidity indicator connected to a chemical humidity detector that detects humidity of the gas exiting the outlet valve.

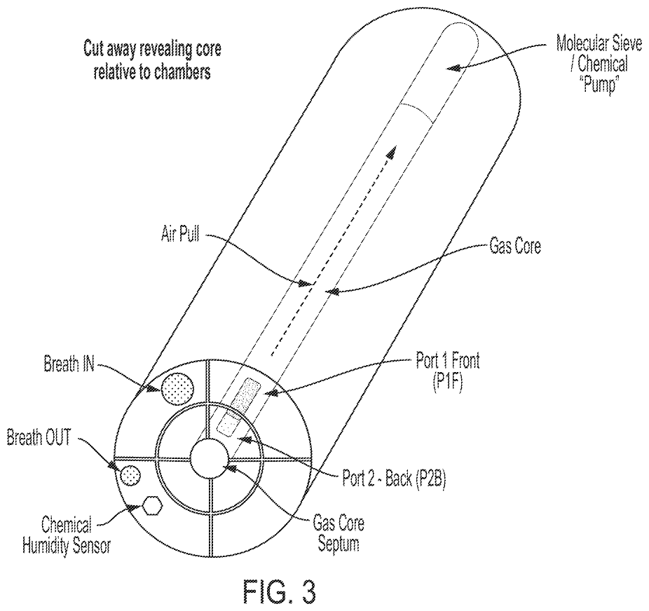

FIG. 4 shows the front of one embodiment of a breath collection/volatile-analyte preservation device and schematically depicts the air flow through an eight (8) chamber device.

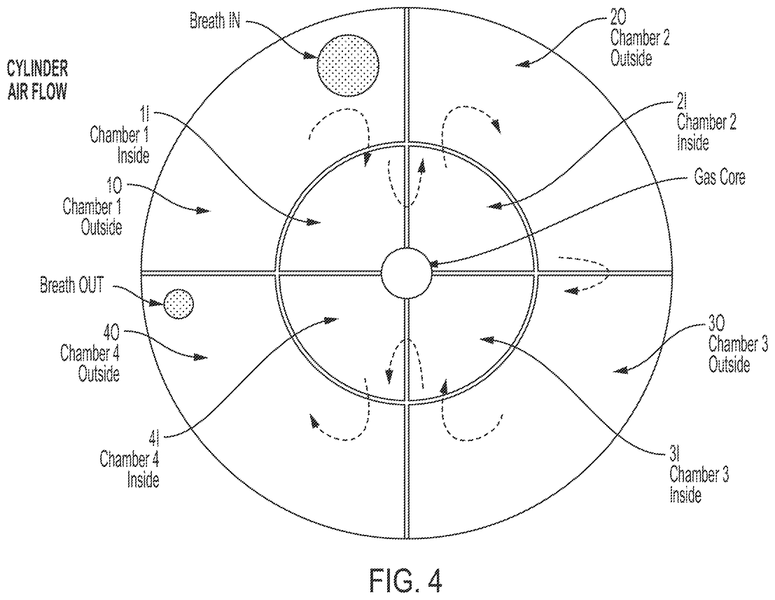

FIG. 5 schematically depicts a cut away of one embodiment of a breath collection/volatile-analyte preservation device showing eight chambers surrounding a central core chamber. The core chamber depicts the direction of airflow by the arrow. The head of the arrow is directed toward a chamber that may contain one or more molecular sieves. The eight chambers are not depicted in the body of the device to more clearly show the central core.

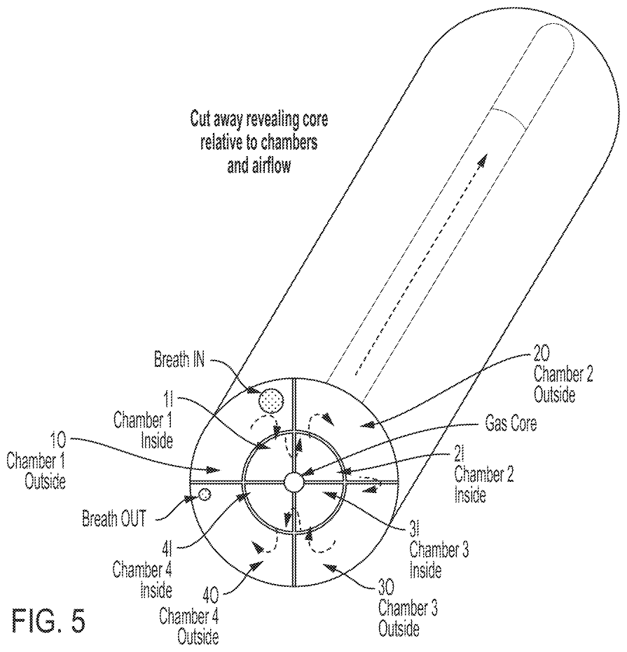

FIG. 6 schematically depicts the configuration of one embodiment of the core.

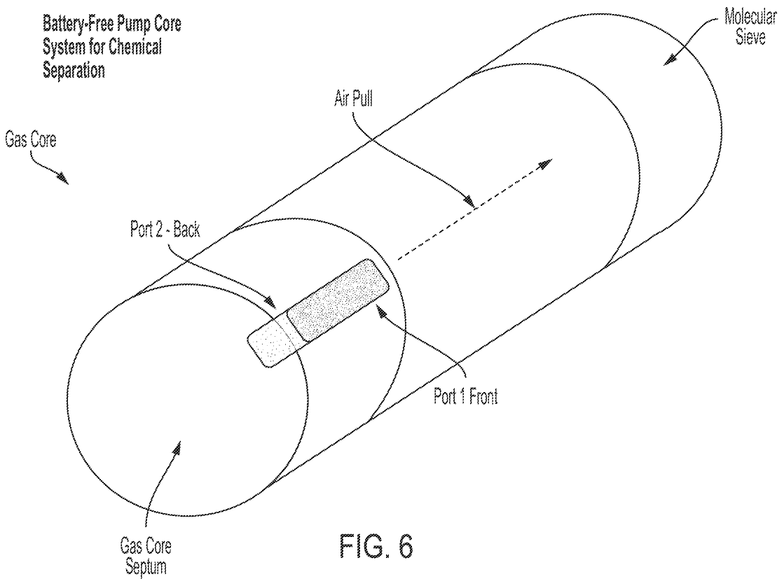

FIG. 7 schematically depicts a central core and three central-core valve positions. The three positions correlate with the valve positions shown in FIG. 2. In Position 1, the valve is shown in the closed position. Position 2 shows both valves open to the exterior of the device. In Position 2, the valve from the collection chamber to the central core (not shown) remains in the closed position. In Position 3, the valve from the collection chamber to the central core is open.

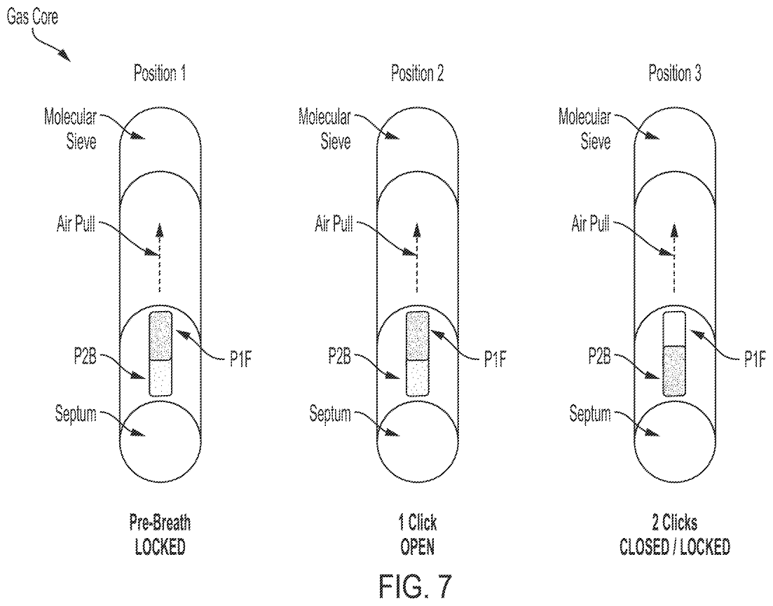

### DETAILED DESCRIPTION

The present invention provides a device that utilizes the composition of exhaled breath, which contains water vapor between 30 to 100% relative humidity (RH), to collect, separate, concentrate, process and otherwise preserve the components and/or analytes contained in exhaled breath. By providing a dry collection chamber comprising materials that have an affinity for water and analytes, including volatile analytes, breath/air is captured as it enters the device and interacts with a variety of materials that bind or retard the flow of different classes of molecules through the collection chamber and/or the central core of the device.

In one embodiment of the present invention, a breath collection device comprises a collection chamber and a core. In one embodiment, the collection chamber comprises a gas and liquid impermeable material; an entry port and a first valve that allows or prevents entry of breath/air into the interior of the device from the exterior of the device; an exit port and a second valve that allows or prevents the exit of breath/air from the interior of the device to the exterior; and a first packing material. In one embodiment, the core comprises a first end, a longitudinal body and a second end, operably connected to the exit port of the collection chamber; a core valve that allows or prevents entry of breath into the core from the collection chamber; a second packing material; and a drying agent in the first end.

In another embodiment of the present invention, a breath collection device comprising a collection chamber a core and a drying agent chamber. In one embodiment, the collection chamber comprises a gas and liquid impermeable material; an entry port and a first valve that allows or prevents entry of breath/air into the interior of the device from the exterior of the device; an exit port and a second valve that allows or prevents the exit of breath/air from the interior of the device to the exterior; and a first packing material. In one embodiment, the core comprises a first end, a longitudinal body and a second end, operably connected to the exit port of the collection chamber; a core valve that allows or prevents entry of breath into the core from the collection chamber; a second packing material; and a drying agent in the first end. In one embodiment of the present invention, the drying agent chamber comprises a longitudinal body operably connected to the core; a valve that allows or prevents entry of breath into the drying agent chamber from the core; and at least one drying agent.

The present invention also provides breath collection kits. In one embodiment, the breath collection kit comprises a collection chamber, a core and a drying agent chamber. In one embodiment, the collection chamber comprises a gas and liquid impermeable material; an entry port and a first valve that allows or prevents entry of breath into the interior of the collection chamber from the exterior of the device; an exit port and a second valve that allows or prevents the exit of breath from the interior of the device to the exterior; and a first packing material. In one embodiment, the core comprises a longitudinal body; a core valve that allows or prevents entry of breath into the core from the collection chamber; and a second packing material. In one embodiment, the drying agent chamber comprises a longitudinal body; a valve that allows or prevents entry of breath into the drying agent chamber from the core; and at least one drying agent.

In another embodiment, the breath collection kit comprises a collection chamber, a core and a drying agent chamber. In one embodiment the collection chamber comprises a gas and liquid impermeable material; an entry port and a first valve that allows or prevents entry of breath into the interior of the collection chamber from the exterior of the device; an exit port and a second valve that allows or prevents the exit of breath from the interior of the device to the exterior; and a first packing material. In one embodiment, the core comprises a longitudinal body; a core valve that allows or prevents entry of breath into the core from the collection chamber; and a second packing material, In one embodiment, the drying agent chamber comprises a longitudinal body; a valve that allows or prevents entry of breath into the drying agent chamber from the core; and at least one drying agent.

The present invention provides a device that provides volatile analytes in a gas phase a high surface area to adsorb/absorb to affinity materials slowing or arresting them in the device. Gases may be collected until the device becomes saturated with water vapor, which is detected at the exit port, allowing for a standardized collection of sample. The collection device is pre-dried, facilitating sample collection. In one preferred embodiment, the gas is breath, which comprises a gas phase, moisture, desired and undesired analytes. The compartments of the collection chamber are arranged to introduce a complex route for the sampled breath to travel, thereby increasing the surface area (and capacity) over which the breath and analytes contact. As used herein, a “desired analyte” is a component of a gas that is targeted for detection, identification, analysis, characterization, quantitation and the like. As used herein, an “undesired analyte” is a component of a gas that is not a “desired analyte.” In one embodiment, the desired analyte is a volatile organic compound (VOC).

In one preferred embodiment, the gas/breath collection/volatile-analyte preservation device of the present invention comprises a collection chamber and a core which may be centrally located in the device.

The collection chamber may be a single chamber or divided into multiple compartments which are operably connected to one another. In one embodiment of the present invention, the collection chamber is divided into two compartments. In another embodiment, the collection chamber comprises four chambers. In yet another and more preferred embodiment, the collection chamber comprises eight or more compartments.

The compartments of the collection chamber are operably connected by openings in the compartments that connect one compartment to another and allow the flow of vapor and/or liquid from one compartment to another. In one embodiment, the openings are arranged to allow unidirectional flow of vapor and/or liquid throughout the collection chamber. In one embodiment, the openings further comprise a valve. In one embodiment, the valve is a one-way valve.

The operably connected openings in the compartments may be arranged to allow the flow of vapor and/or liquid over substantially the entire length of a compartment. In one embodiment, each compartment comprises one opening that allows vapor and/or liquid to enter the compartment from a first adjacent, connected compartment and another opening that allows the vapor and/or liquid to exit the compartment to a second adjacent, connected compartment.

The collection chamber contains a first compartment that is operably connected to the exterior of the device by a port to allow breath/air to enter the collection chamber (entry port) and second compartment that is operably connected to the exterior of the device by a port to allow breath/air to exit the collection chamber (exit port). In one embodiment where there is a single compartment in the collection chamber, there are two openings to the exterior of the single-compartment collection device.

In one embodiment, the closing of the valves at the entry and exit ports of the device occurs simultaneously with the opening of the valve on the core. In a preferred embodiment, the closing of the valves at the entry and exit ports to the exterior of the device precede the opening of the valve on the core to the collection chamber.

In one embodiment, the device further comprises a chemical detector that senses the presence of water vapor at the exit port. In a further embodiment, the device further comprises an indicator observable from the exterior of the device that is operably linked to the chemical detector.

Both the interior and exterior structures of the device are constructed to be impermeable to gas and liquid. In a preferred embodiment, the liquid impermeable material comprises a non-corrosive material. In one embodiment, the non-corrosive material comprises a metal. In a more preferred embodiment the metal is stainless steel. In another embodiment, the non-corrosive material is not structurally rigid.

In another embodiment, the devices of the present invention are substantially free of leachable organic compounds. As used herein, “leachable organic compounds” are compounds that migrate into a breath sample when present in any one or more components of within the devices of the present invention. Plastics are a common source “leachable organic compounds” and in certain embodiments, the devices of the present invention do not comprise plastics. As used herein, “substantially free of leachable organic compounds” means that one or more “leachable organic compounds” do not interfere with the detection of one or more desired analytes.

In another embodiment, the collection chamber is substantially free of leachable organic compounds. In yet another embodiment, the core is substantially free of leachable organic compounds. In a further embodiment, the drying agent chamber is substantially free of leachable organic compounds.

In another embodiment, one or more interior surfaces of the devices of the present invention comprise the non-corrosive material comprises polyvinyl fluoride. In one embodiment, the polyvinyl fluoride is in the form of a film, coating or lining or the like. Polyvinyl fluoride is commercially available in many forms including under the tradename Tedlar®.

The devices of the present invention comprise at least two ports comprising a first valve that allows entry of breath/air into the interior of the device from the exterior of the device in certain valve positions and a second port comprising a second valve that allows the exit of breath/air from the interior of the device to the exterior. In certain other valve positions, the valves prevent either the entry or exit of liquid or gas to or from the device.

The core may be operably connected to a compartment of the collection chamber. In one embodiment, the core is separated from the collection chamber by a valve. In certain other valve positions, the valve prevents the entry of liquid or gas to the core from the collection chamber.

In one embodiment, the core comprises a first end, a longitudinal body and a second end. The longitudinal body further comprises a first compartment and a second compartment. In one embodiment the first compartment comprises the first end. The first compartment further comprises one or more drying agents. In a preferred embodiment, a drying agent is selected from silica, activated charcoal, calcium sulfate, calcium chloride, molecular sieves, alcohols, and acetones. In a most preferred embodiment, the drying agent is one or more molecular sieves.

In yet another embodiment, the core does not comprise a first and second compartment wherein one of the compartments comprises one or more drying agents. In another embodiment, the breath collection device comprises a drying agent chamber comprising a longitudinal body and a valve that allows or prevents entry of breath into the drying agent chamber from the core when the drying agent chamber is operably connected to the core.

In yet another embodiment, the core further comprises a port and a valve that is positioned substantially at the second end and is operably connected to a compartment of the collection chamber. In yet another embodiment, the core further comprises a septum at the second end of the core.

The additional element of a sealed core provides a source of “chromatographic energy” upon the closure of the device to the external environment (i.e. closing of the valves at the entry and exit ports). In certain embodiments, upon closure to the external atmosphere, a valve opens to the core of the device thus connecting the collection chamber to the core.

The core may or may not be pre-charged under vacuum to rapidly draw any saturated water to a hydrophobic filter, limiting/controlling the rate of vacuum discharge. This may be used as a force to assist in capillary chromatography separations. As water vapor is transferred by capillary and gas phase chemistry toward the molecular sieve (or equivalent reagent) at the first end of the core, a gradient of water vapor may be used as a “mobile phase” to further separate materials.

Additionally, in certain embodiments, the molecular sieve at the first end of the core provides a gradient of water vapor (>1000 fold) from the second end to the first end. The controlled rate of water flow (approximately 6 ml per 5 L of breath) allows for a significant amount of “mobile phase” to move over the packing material of the core. Finally, in certain embodiments, as the water vapor is moved fully from the collection chamber through the core and into the molecular sieve, any bacteria or other contaminants are prevented from reaching the molecular sieve, so degradation of the sieve is prevented, thus providing a bio-static compartment for indefinite ambient storage of samples.

Upon closing of the device to the external environment, and opening of the core to the collection chamber, the core exposes the collection chamber to a strong pressure gradient (from ˜760 mm Hg to <7 mm Hg->100 fold) and a strong gradient of water vapor (from 100% relative humidity to <0.1% relative humidity (greater than a 1000-fold concentration difference)). Using gradients generated by the core placed under negative pressure combined with the gradient-producing effect of the molecular sieves at the first end of the core, and further combined with packing materials of different pore sizes, allows the application of capillary and affinity chromatography in a contained device without external power.

In one embodiment, the materials packing (“packing material”) the collecting chamber (“first packing material”) and core (“second packing material”) may be arranged such that the materials packing the core have a higher affinity for desired analytes in breath/air than the materials packing the collection chamber.

In one embodiment, the first packing material comprises material with greater affinity for at least one desired analyte in a gas phase compared to an undesired analyte. In another embodiment, the first packing material comprises a hydrophilic material. In yet another embodiment, the first packing material comprises a hydrophobic material.

In one embodiment, at least one desired analyte has a with greater affinity for the second packing material compared to the gas phase. In another embodiment, the second packing material comprises a hydrophilic material. In yet another embodiment, the second packing material comprises a hydrophobic material. In yet another embodiment, the second packing material comprises a sorbent. As used herein, “sorbent” means a material which has the property of collecting another substance by the process of sorption. A wide variety of commercial sorbents are available for use with techniques such as thermal desorption. A sorbent suitable for use in the present invention should retain and desorb desired analytes; allow heating to temperatures in excess of that required for desorption; generate minimal or no artifacts on heating; have a low affinity for water; and low metal content. Commercial sorbents include porous polymers (e.g. Tenax® TA), graphitized carbon black (e.g. Carbopack™), carbon molecular sieves (e.g. Carboxen®), and inorganic adsorbents (e.g. silica gel, molecular sieves and glass wool), and the like.

In another embodiment, the first and/or second packing materials may be further functionalized chemically to add, mask, modify or otherwise augment the chemical functionality and/or affinity of the first and/or second packing material for a desired analyte or an undesired analyte.

The first and second packing materials may be arranged in the collection chamber and core in a manner to enrich portions of the collection chamber and/or core in one or more desired analytes. In one embodiment, for example, the materials packing the core may be arranged such that packing materials in the core have a higher affinity for desired analytes in breath/air than the materials packing the collection chamber. Such an arrangement of packing materials allows for the distribution of analytes across and throughout the collection chamber and core depending on the particular analyte and its affinity for various materials. Other arrangements of the first and second packing materials are also contemplated by the present invention

In one embodiment, the first packing material comprises material with greater affinity for at least one desired analyte compared to the second packing material. In another embodiment, the first packing material comprises material with less affinity for at least one desired analyte compared to the second packing material. In yet another embodiment, at least one portion of the core comprises a sorbent that retains and desorbs a desired analyte. In one embodiment, the desired analyte desorbs from the sorbent upon heating.

Once water vapor has been transferred to the core, the container is now a “gas-phase” chemistry environment. As such, gas phase chemistries come to much faster equilibrium reactions than liquid environments. Thus, placing different affinity matrices sequentially in the device allows molecules to move from the collection chamber to the core using gas-phase chemistries. Finally, as the device reduces or eliminates water, the interior of the device becomes viable for anhydrous chemistries that can be used to facilitate the detection of other molecules.

The compartments comprising the collection chamber preferably contain packing materials that increase the surface area of the collection device and capacity of the device for analytes. In a preferred embodiment, the collection chamber comprises packing material that will trap water vapor and volatile analytes. In one preferred embodiment, the packing material is molecular trap paper. In another embodiment, the packing material comprises filter paper or other absorbent paper. In another embodiment, the material is folded, ground, or otherwise compressed to maximize the surface area of the material while maintaining air and vapor flow. In another embodiment, the collection chamber contains a packing material comprising one or more zeolites.

In certain embodiments of the present invention, the materials contained in the collection chamber, such as molecular trap paper, are dried to a relative humidity of 20% and more preferably 10% or lower. Dry adsorbant material, such as molecular trap paper, allows for rapid adsorption of water vapor as the sampled breath travels through a complex circuit, maximizing the interaction time with each compartment. The dried material ensures that breath is collected until the device is saturated (or set for an appropriate humidity level using an indicator), thus ensuring an equivalent amount of sample regardless of the tidal volume of the provider. In certain embodiments, a simple chemical humidity sensor at the vent portion of the device indicates when saturation is achieved. Once this occurs, a standard amount of sample material is collected within the devices of the present invention.

In certain embodiments, the device contains materials of different affinities for different classes of molecules. Therefore, once water is adsorbed and absorbed, volatiles that separate will be trapped by affinity materials.

In one embodiment, the relative humidity in the collection chamber, core and the materials contained therein, through the combined forces of the pressure and vapor gradients created by the core, is reduced to below 10%, 5%, 3%, 2% or 1%. In a preferred embodiment, the humidity of the collection chamber, core and materials contained therein is reduced to below 0.5%. In a most preferred embodiment, the humidity of the collection chamber, core and materials contained therein is reduced to below 0.1%.

Upon drying, the integrity of analytes are protected from degradation reactions that are associated with water. Further, the device is biologically static as no organisms may grow without water. Filtration of water and water vapor to the molecular sieve provides a physical barrier that maintains sterility of the sieve material, preventing any type of breakdown of the sieve material. Thus, the device also extends the integrity of the sample in a room-temperature environment.

The present invention also provides a method of collecting exhaled breath and/or air and processing, detecting and analyzing one or more analytes contained therein.

In one embodiment, the present invention provides a method of collecting an exhaled breath sample comprising the steps of exhaling breath into a device of the present invention followed by closing of valves at the entry and exit ports prior to the opening of the core valve and detecting one or more analytes.

In one embodiment, analytes are detected by sampling gas from core, desorption from core material by heat or other means. In one embodiment, the analytes are detected by first removing a separable portion of the core from the core followed by thermal desorption, extraction or other forms of desorption of the adsorbed analyte. In another embodiment, the separable portion of the core is a thermal desorption tube comprising at least one analyte adsorbed to a sorbent. In one embodiment, the thermal desorption tube is essentially free of water and is coupled to a gas chromatograph and analytes are detected and identified by mass spectrometry.

In one embodiment, the methods of the present invention detect one or more desired analytes in a portion of the collection chamber. In another embodiment, one or more desired analytes are detected in a portion of the core. In another embodiment, the portion of the collection chamber is separable from the collection chamber. In another embodiment, the portion of the core is separable from the core.

In one embodiment of the methods of the present invention, exhalation of breath into a device comprises multiple exhalations. In another embodiment, the exhalation of breath into a device comprises a single breath.

In one embodiment of the methods of the present invention, the opening of the core valve causes drying of one or more compartments of the collecting chamber. It will be understood that the drying may proceed until the relative humidity (RH) inside the collection chamber attains a level between 10 and 20%, between 5 and 10%, between 0.1 and 1% or below about 0.1%.

In another embodiment, the methods of collecting an exhaled breath sample of the present invention comprise the steps of: a) providing a breath collection kit of the present invention comprising a collection chamber, core and drying agent chamber; b) exhaling breath into the collection chamber followed by closing of valves at the entry and exit ports; c) operably connecting the core to the collection chamber either prior to or following step b); d) operably connecting the drying agent chamber to the core either prior to or following step c); and e) opening the valves to the collection chamber, core and drying agent chamber.

In another embodiment, the methods of collecting an exhaled breath sample of the present invention comprise the steps of: a) providing a breath collection kit of the present invention comprising a collection chamber and core; b) exhaling breath into the collection chamber followed by closing of valves at the entry and exit ports; c) operably connecting the core to the collection chamber either prior to or following step b); and d) opening the valves to the collection chamber and core.

In another embodiment of the present invention, the methods of collecting an exhaled breath sample comprise opening the valves to the collection chamber, core and drying agent chamber after a period of at least 30 minutes following exhaling breath into the collection chamber.

In another embodiment of the present invention, the methods of collecting an exhaled breath sample comprise opening the valves to the collection chamber and core after a period of at least 30 minutes following exhaling breath into the collection chamber.

In another embodiment, the methods of collecting an exhaled breath sample further comprise the step of detecting one or more analytes from a portion of the core. In certain embodiments, the portion of the core is separable from the core. In one embodiment, the one or more analytes are detected by gas chromatography-mass spectrometry (GC-MS).

In yet another embodiment, the present invention provides a method of collecting an exhaled breath sample comprising the steps of: a) providing a collection chamber comprising a gas and liquid impermeable material; an entry port and a first valve that allows or prevents entry of breath into the interior of the collection chamber from the exterior of the device; an exit port and a second valve that allows or prevents the exit of breath from the interior of the device to the exterior; and a first packing material; and b) exhaling breath into the collection chamber followed by closing of valves at the entry and exit ports.

In yet another embodiment, the present invention provides a method of drying a gas comprising the steps of: a) providing a collection chamber comprising a gas and liquid impermeable material; an entry port and a first valve that allows or prevents entry of breath into the interior of the collection chamber from the exterior of the device; an exit port and a second valve that allows or prevents the exit of breath from the interior of the device to the exterior; and a first packing material; b) injecting the gas into the collection chamber followed by closing of valves at the entry and exit ports; and c) exposing the gas to the first packing material for a period of time sufficient to reduce the moisture content of the gas.

In yet another embodiment, the present invention provides a method of enriching a gas with a desired analyte comprising the steps of: a) providing a collection chamber comprising a gas and liquid impermeable material; an entry port and a first valve that allows or prevents entry of breath into the interior of the collection chamber from the exterior of the device; an exit port and a second valve that allows or prevents the exit of breath from the interior of the device to the exterior; and a first packing material; b) injecting the gas into the collection chamber followed by closing of valves at the entry and exit ports; and c) exposing the gas to the first packing material for a period of time sufficient to reduce the moisture content of the gas and remove a greater amount of undesired analytes from the gas compared to desired analytes.

In yet another embodiment, the present invention provides a method of enriching a gas with a desired analyte comprising the steps of: a) exhaling breath into a device of the present invention comprising a collection chamber, a core and optionally a drying agent chamber; b) followed by closing of valves at the entry and exit ports prior to the opening of the core valve; and c) exposing the gas to the first packing material and the second packing material for a period of time sufficient to reduce the moisture content of the gas and remove a greater amount of undesired analytes from the gas compared to desired analytes.

In yet another embodiment, the present invention provides a method of enriching a desired analyte in a gas comprising the steps of: a) exhaling breath into a device of the present invention comprising a collection chamber, a core and optionally a drying agent chamber; b) followed by closing of valves at the entry and exit ports prior to the opening of the core valve; and c) exposing the gas to the first packing material and the second packing material for a period of time sufficient to adsorb a desired analyte to a portion of the collection chamber or core to a greater degree compared to an undesired analyte, thereby enriching the desired analyte.

What is claimed is:

1. A breath collection device comprising:
(a) a collection chamber comprising
a gas and liquid impermeable material;
an entry port and a first valve that allows or prevents entry of breath/air into the interior of the device from the exterior of the device;
an exit port and a second valve that allows or prevents the exit of breath/air from the interior of the device to the exterior; and
a first packing material;
(b) a core comprising
a first end, a longitudinal body and a second end, operably connected to the exit port of the collection chamber;
a core valve that allows or prevents entry of breath into the core from the collection chamber;
a second packing material; and
a drying agent in the first end.

2. The breath collection device of claim 1, wherein the first packing material in the collection chamber is dried to reduce the relative humidity (RH) in the collection chamber to about 20%.

3. The breath collection device of claim 2, wherein the first packing material in the collection chamber is dried to reduce the RH in the collection chamber to about 10%.

4. The breath collection device of claim 1, wherein the first packing material comprises molecular trap paper.

5. The breath collection device of claim 1, wherein the first packing material is dried.

6. The breath collection device of claim 1, wherein the collection device comprises one or more compartments.

7. The breath collection device of claim 6, wherein the collection device comprises two, three, four, five six seven or eight separate compartments.

8. The breath collection device of claim 7, wherein the compartments of the collection chamber are operably connected by openings in the compartments that connect one compartment to another and allow the flow of vapor and/or liquid from one compartment to another.

9. The breath collection device of claim 8, wherein the compartments comprise an opening arranged to allow unidirectional flow of vapor and/or liquid throughout the compartments of the collection chamber.

10. A method of collecting an exhaled breath sample comprising the steps of:
a) exhaling breath into the device of claim 1;
b) followed by closing of valves at the entry and exit ports occur prior to the opening of the core valve; and
c) detecting one or more analytes.

11. The method of claim 10, wherein the opening of the core valve causes drying of one or more compartments of the collecting chamber.

12. The method of claim 11, wherein the drying produces a relative humidity (RH) of between 10 and 20% in the collecting chamber.

13. A breath collection device comprising:
(a) a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material;
(b) a core comprising a longitudinal body operably connected to the collection chamber;
a core valve that allows or prevents entry of breath into the core from the collection chamber; and
a second packing material; and
(c) a drying agent chamber comprising
a longitudinal body operably connected to the core;
a valve that allows or prevents entry of breath into the drying agent chamber from the core; and
at least one drying agent.

14. A method of collecting an exhaled breath sample comprising the steps of:
a) exhaling breath into the device of claim 13;
b) followed by closing of valves at the entry and exit ports prior to the opening of the core valve; and
c) detecting one or more analytes.

15. A breath collection kit comprising:
(a) a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material;
(b) a core comprising
a longitudinal body;
a core valve that allows or prevents entry of breath into the core from the collection chamber; and
a second packing material; and
(c) a drying agent chamber comprising
a longitudinal body;
a valve that allows or prevents entry of breath into the drying agent chamber from the core; and
at least one drying agent.

16. The breath collection kit of claim 15, wherein at least one portion of the core is separable from the core.

17. A breath collection kit comprising:
a) a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material;
b) a core comprising
a first end, a longitudinal body and a second end;
a core valve that allows or prevents entry of breath into the core from the collection chamber;
a second packing material; and
a drying agent in the first end.

18. The breath collection kit of claim 17, wherein at least one portion of the core is separable from the core.

19. A method of collecting an exhaled breath sample comprising the steps of:
a) providing a kit of claim 15;
b) exhaling breath into the collection chamber followed by closing of valves at the entry and exit ports;
c) operably connecting the core to the collection chamber either prior to or following step b);
d) operably connecting the drying agent chamber to the core either prior to or following step c); and
e) opening the valves to the collection chamber, core and drying agent chamber.

20. A method of collecting an exhaled breath sample comprising the steps of:
a) providing a kit of claim 17;
b) exhaling breath into the collection chamber followed by closing of valves at the entry and exit ports;
c) operably connecting the core to the collection chamber either prior to or following step b); and
d) opening the valves to the collection chamber and core.

21. The method of claim 19, wherein step e) is performed after a period of at least 30 minutes following performance of step a).

22. The method of claim 20, wherein step e) is performed after a period of at least 30 minutes following performance of step a).

23. The method of claim 19 comprising detecting one or more analytes from the portion of the core separated from the core.

24. The method of claim 20, wherein the one or more analytes are detected by gas chromatography-mass spectrometry (GC-MS).

25. A method of collecting an exhaled breath sample comprising the steps of:
a) providing a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material; and
b) exhaling breath into the collection chamber followed by closing of valves at the entry and exit ports.

26. A method of drying a gas comprising the steps of:
a) providing a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material;
b) injecting the gas into the collection chamber followed by closing of valves at the entry and exit ports; and
c) exposing the gas to the first packing material for a period of time sufficient to reduce the moisture content of the gas.

27. A method of enriching a gas with a desired analyte comprising the steps of:
a) providing a collection chamber comprising
a gas and liquid impermeable material;
an entry port and a first valve that allows or prevents entry of breath into the interior of the collection chamber from the exterior of the device;
an exit port and a second valve that allows or prevents the exit of breath from the interior of the device to the exterior; and
a first packing material;
b) injecting the gas into the collection chamber followed by closing of valves at the entry and exit ports; and
c) exposing the gas to the first packing material for a period of time sufficient to reduce the moisture content of the gas and remove a greater amount of undesired analytes from the gas compared to desired analytes.

28. A method of enriching a gas with a desired analyte comprising the steps of:
a) exhaling breath into the device of claim 1;
b) followed by closing of valves at the entry and exit ports prior to the opening of the core valve; and
c) exposing the gas to the first packing material and the second packing material for a period of time sufficient to reduce the moisture content of the gas and remove a greater amount of undesired analytes from the gas compared to desired analytes.

29. A method of enriching a desired analyte in a gas comprising the steps of:
a) exhaling breath into the device of claim 1;
b) followed by closing of valves at the entry and exit ports prior to the opening of the core valve; and
c) exposing the gas to the first packing material and the second packing material for a period of time sufficient to adsorb a desired analyte to a portion of the collection chamber or core to a greater degree compared to an undesired analyte, thereby enriching the desired analyte.
